(19) United States
(12) Patent Application Publication
(10) Pub. No.: US 2009/0274775 A1
(43) Pub. Date: Nov. 5, 2009

(54) Lithium combinations, and uses related thereto

(75) Inventor: Philip Maxwell Satow, New York, NY (US)

Correspondence Address: FOLEY AND LARDNER LLP;SUITE 500, 3000 K STREET NW, WASHINGTON, DC, 20007, US

(73) Assignee: NOVEN THERAPEUTICS, LLC

(21) Appl. No.: 12/314,022

(22) Filed: Dec. 2, 2008

Related U.S. Application Data
(63) Continuation of application No. 11/108,476, filed on Apr. 18, 2005, now abandoned.
(60) Provisional application No. 60/563,347, filed on Apr. 19, 2004.

(51) Int. Cl. A61K 33/00; A61P 25/00

(52) U.S. Cl. 424/715; 424/722

(57) ABSTRACT

The present invention relates to combinatorial therapies for treating anxiety, depression or psychotic conditions using a lithium salt and a psychoactive drug selected from the group consisting of serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, a sedative-hypnotic drug, and an atypical antipsychotic.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The present invention relates to pharmaceutical products used in the treatment of various psychological conditions. More particularly, the invention relates to combination therapy of a pharmaceutically acceptable lithium salt and another pharmaceutically active agent. In preferred embodiments the invention relates to combination formulations of a lithium salt and the other pharmaceutically active agent. In most preferred embodiments the invention relates to dosage forms which can be administered on a once daily basis.

### BACKGROUND OF THE INVENTION

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions.

Treatment regimens commonly include the use of tricyclic antidepressants, monoamine oxidase inhibitors, some psychotropic drugs, lithium carbonate, and electroconvulsive therapy (ECT) (see R J. Baldessarini in Goodman & Gilman's The Pharmacological Basis of Therapeutics, 9th Edition, Chapter 19, McGraw-Hill, 1996 for a review). More recently, new classes of antidepressant drugs are being developed including selective serotonin reuptake inhibitors (SSRIs), specific monoamine reuptake inhibitors and 5-HT_{1A} receptor agonists, antagonists and partial agonists.

Anxiety is an emotional condition characterized by feelings such as apprehension and fear accompanied by physical symptoms such as tachycardia, increased respiration, sweating and tremor. It is a normal emotion but when it is severe and is disabling it becomes pathological.

Anxiety disorders are generally treated using benzodiazepine sedative-antianxiety agents. Potent benzodiazepines are effective in panic disorder as well as in generalized anxiety disorder, however, the risks associated with drug dependency may limit their long-term use. 5-HT_{1A} receptor partial agonists also have useful anxiolytic and other psychotropic activity, and less likelihood of sedation and dependence (see R J. Baldessarini in Goodman & Gilman's The Pharmacological Basis of Therapeutics, 9th Edition, Chapter 18, McGraw-Hill, 1996 for a review).

Bipolar Disorder is a psychiatric condition which is prevalent across cultures and age groups. The lifetime prevalence of Bipolar Disorder can be as high as 1.6%. DSM-IV, p. 353 (American Psychiatric Association, Washington, D.C. 1997). Bipolar Disorder is a recurrent disorder characterized by one or more Manic Episodes immediately before or after a Major Depressive Episode or may be characterized by one or more Major Depressive Episodes accompanied by at least one Hypomanic Episode. Additionally, the symptoms must cause clinically significant distress or impairment in social, occupational, or other important areas of functioning.

In some cases the Hypomanic Episodes themselves do not cause impairment; however, the impairment may result from the Major Depressive Episodes or from a chronic pattern of unpredictable mood episodes and fluctuating unreliable interpersonal and occupational functioning. The symptoms of Bipolar Disorder must not be better accounted for by a psychotic condition or due to the direct physiological effects of a medication, other somatic treatments for depression, drugs of abuse, or toxin exposure.

Bipolar Disorder is associated with a significant risk of completed suicide. Further, the patient suffering from Bipolar Disorder is likely to suffer from school truancy, school failure, occupational failure, or divorce.

Therefore, Bipolar Disorder is a serious, fairly prevalent, psychological condition which is clearly distinguished from psychotic conditions such as schizophrenia. DSM-IV, p. 353 (American Psychiatric Association, Washington, D.C. 1994). DSM-IV, p. 353 (American Psychiatric Association, Washington, D.C. 1994).

There remains a long felt need for treatments which provide a favorable safety profile and effectively provide relief for the patient suffering an anxiety, depression or psychotic condition.

### OBJECT OF THE INVENTION

It is an object of the invention to provide a combination therapy for the treatment of various psychological conditions.

It is another object of the invention to provide combination therapy of psychological conditions on a once daily basis.

It is still another object of the invention to provide a combination therapy for the treatment of psychological conditions that includes a lithium salt component.

It is yet another object of the invention to provide a combination therapy for the treatment of psychological conditions that includes a once daily lithium salt component.

An even further object of the invention is to provide a combination therapy for psychological conditions that includes a lithium component and a selective serotonin reuptake inhibitor (SSRI) component.

It is still another object of the invention to provide fixed combination products for the treatment of psychological conditions containing a lithium component and an SSRI component.

An even further object of the invention is to provide a fixed combination product suitable for once daily administration containing a lithium salt and an SSRI component.

A still further object of the invention is to provide a synergistic combination of a lithium salt and a psychoactive drug other than lithium.

Yet another object of the invention is to provide a synergistic fixed combination of a lithium salt and an SSRI.

An even further object of the invention is to provide a combination therapy of a psychological condition with dosages of the component active agents which would be sub-therapeutic if one of the active agents were used alone.

Still another object of the invention is to help prevent precipitating a manic episode in a patient being treated for depression.

Yet another object of the invention is to provide a method of using lithium salts in lessening or preventing the risk of suicide resulting from the use of a non-lithium psychoactive drug.

Still further objects of the invention will be apparent to those of ordinary skill.

### SUMMARY OF THE INVENTION

The foregoing objects are achieved by providing a co-therapy regimen of a lithium salt and another pharmaceutically active agent which may be in the form of a free base, a free acid, or a pharmaceutically acceptable salt thereof, or a neutral compound. The present invention relates especially to methods and compositions for treating patients suffering from an anxiety, depression or psychotic disorder. In particular, the invention contemplates co-administering lithium salts in combination with a second psychoactive active drug selected from a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, or a sedative-hypnotic drug. For purposes of the present invention, unless clearly indicated by the context to the contrary, any reference made to an acidic drug includes the pharmaceutically acceptable salts thereof, any reference made to a basic drug includes the pharmaceutically acceptable acid addition salts thereof, and any reference to any drug includes the various polymorphs, solvents, optical isomers, and racemic or diasteromeric mixtures as well. Where cis- and trans-isomers exist, the diastereomeric mixture thereof is intended to include each of the separate isomers. Where one of the cis- or trans-isomers is mentioned, it is intended to include the other as well as mixture thereof. In any situation where optical isomers or diasteriomers exist, mixtures thereof in any ratio is contemplated, whether or not naturally occurring.

In certain preferred embodiments, the lithium is provided in an amount ranging from 150 mg to 2000 mg per day. In certain preferred embodiments, the lithium component is provided in suitable dosages for prophylaxis or acute treatment, i.e. 150 mg to 1200 mg/day in the prophylaxis and up to 2000 mg/day in the acute treatment of states of mania. In certain preferred embodiments, the lithium is provided in an amount ranging from 300 mg to 900 mg and even more preferably 600 mg to 900 mg

In certain preferred embodiments, the lithium is provided in a slow release preparation, e.g., to maintain stable lithium plasma levels over the course of about 6, about 8, about 12, about 18, or even about 24 hours or longer. Exemplary forms of lithium salts include, without limitation, lithium carbonate, lithium citrate, lithium acetate, lithium glutamate, lithium orotate, lithium thionate and lithium sulphate. Other salts of lithium (both inorganic and organic) are also suitable so long as the particular salt in question is pharmaceutically acceptable. Those of ordinary skill in the art will be able to select alternative salts within this group as desired.

In certain preferred embodiments, the lithium is provided in a once-a-day formulation.

In certain embodiments, the lithium is co-administered with a serotonin reuptake inhibitor (SRI). In certain preferred embodiments, the SRI is a selective serotonin reuptake inhibitor (SSRI), such as a fluoxetinoid (fluoxetine, norfluoxetine, etc.) or a nefazodonoid (nefazodone, hydroxynefazodone, oxonefazodone, etc.). Other exemplary non-limiting SSRI's include duloxetine, venlafaxine, milnacipran, citalopram, escitalopram, fluvoxamine, paroxetine and sertraline.

In certain embodiments, the lithium is co-administered with a sedative-hypnotic drug, such as selected from the group consisting of a benzodiazepine (such as alprazolam, chlordiazepoxide, clonazepam, chlorazepate, clobazam, diazepam, halazepam, lorazepam, oxazepam and prazepam, etc.), zolpidem, and barbiturates.

In certain embodiments, the lithium is co-administered with a 5-HT_{1A} receptor partial agonist, such as selected from the group consisting of buspirone, flesinoxan, gepirone and ipsapirone.

In certain embodiments, the lithium is co-administered with a norepinephrine reuptake inhibitor, such as selected from tertiary amine tricyclics and secondary amine tricyclics, as well as nisoxetine, atomoxetine, etc. Exemplary tertiary amine tricyclics include amitriptyline, clomipramine, doxepin, imipramine and trimipramine. Exemplary secondary amine tricyclics include amoxapine, desipramine, maprotiline, nortriptyline and protriptyline.

In certain embodiments, the lithium is co-administered with a monoamine oxidase inhibitor, such as selected from the group consisting of isocarboxazid, phenelzine, tranylcypromine, selegiline and moclobemide.

In certain embodiments, the subject method is treating a patient suffering from or susceptible to Bipolar Disorder, Bipolar Depression or Unipolar Depression.

Another aspect of the present invention provides a packaged pharmaceutical comprising: (i) a first member which is a mood-stabilizing lithium formulation, and (ii) at least one second member which is a drug selected from the group consisting of a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, and a sedative-hypnotic drug; and (iii) and a label indicating the use of the packaged pharmaceutical for use in the treatment of a patient suffering from an anxiety, depression or psychotic disorder. The packaged pharmaceutical may optionally contain other pharmaceutically active compounds so long as they are not incompatible with the other components when coadministered or used in cotherapy. The various active agents may be contained in separate formulations, which may be used in cotherapy at different times during the day or coadministered simultaneously, or separately, in any sequence, or the various agents may be in combination formulations, or if there are more than two active agents contemplated, one or more may be in separate formulations (or separate combination formulation) and the remaining active agents may be combined in one formulation. Those of ordinary skill in the art will recognize many variations of this theme and will be able to prepare and use such variation in accordance with the present invention.

In certain embodiments, the lithium formulation and one or more of the second drug are commingled in a single dosage form. In other embodiments, the lithium formulation and the second drug are provided in separate dosage forms.

In certain preferred embodiments, the packaged pharmaceutical is formulated for oral administration. In a further preferred embodiment, there is provided a single oral dosage formulation of a sustained release lithium carbonate and a at least one second component selected from a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, or a sedative-hypnotic drug.

The one or more members making up the second component may be in single daily dosage form or multiple daily dosage form. When the lithium component and one or more of the other active agents are combined in a single dosage form, each of the active agents are formulated to be administered in the same dosage regimen (i.e. once daily, twice daily, three times daily, etc.) as will be appreciated by those of ordinary skill. Alternatively, where the lithium is in a once daily formulation and one or more of the other active agents are in a multiple times per day formulation, the formulations may still be combined into a fixed combination dosage form provided that the total of lithium content in such combined formulation dosage unit is a fractional part of the total daily dose of lithium intended to be delivered. For example, where 1200 mg of lithium is intended to be delivered per day together with another active agent, both agents are generally formulated to be in the same regimen (once daily, twice daily, etc.). Alternatively, the lithium can be in a once daily format while the other active agent can be in a twice daily format provided that the amount of lithium in a single dosage unit is ½ the daily dose so that on administration of one dosage unit twice daily, the full daily dose of lithium and the other active agent in the fixed combination is still achieved. The same can be done when the other active agent in the fixed combination requires a three or four times a day administration provided that the lithium content of a single dosage unit is ⅓ or ¼ of the total daily dose of lithium respectively. It should also be noted that due to volume constraints in the size of the tablet or capsule, it may be desirable to have the total daily dosage dispensed from two or more fractions of the total daily dose, even if administered at the same time. Thus, for example a once daily formulation of a total daily dose of 1200 mg of the lithium compound and a total daily dose of 100 mg of another agent may be administered as two capsules or two tablets each containing 600 mg of the lithium compound and 50 mg of the other agent, each in a once daily format. In all of the above, if desired, the amounts indicated for a single dosage unit may be fractionated into two or more equal subfractional dosage units which would then require two or more dosage units to be taken at any one time. (i.e., the of the daily dose example above can actually be dosage units having ½ of the daily dose so that 4 dosage units might be taken at one time (once-daily administration), two dosage units taken twice daily, or 1 dosage unit 4 times a day.) Similarly, if the lithium component is in a twice daily format and the second drug is a once daily administration format, the fixed dosage combinations of the present invention can still be utilized provided that ½ of the total daily dose of the second drug is administered twice daily along with the lithium. Other variation on these themes will be appreciated by those of ordinary skill.

In certain preferred embodiments, the lithium formulation and the second drug are formulated for once-a-day administration.

Yet another aspect of the present invention provides a kit comprising: (i) a lithium salt formulation, and a second drug selected from the group consisting of a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, and a sedative-hypnotic drug; and (ii) instructions for co-therapy using and/or for co-administering the lithium formulation and the second drug in a treatment of a patient suffering from an anxiety, depression or psychotic disorder.

Still another aspect of the invention provides a method for preparing a pharmaceutical preparation, comprising combining

(i). a pharmaceutically acceptable lithium compound,

(ii). a second drug selected from the group consisting of a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, and a sedative-hypnotic drug, and

(iii). At least one pharmaceutically acceptable excipient in a composition for simultaneous administration of the lithium formulation and the second drug.

The present invention also provides a method for conducting a pharmaceutical business, comprising manufacturing a packaged pharmaceutical or kit as described herein; and (i) marketing to healthcare providers the benefits of using the packaged pharmaceutical or kit in the treatment of a patient suffering from an anxiety, depression or psychotic disorder; and/or (ii) providing a distribution network for selling the packaged pharmaceutical or a kit, along with providing instruction material to patients or physicians for using the packaged pharmaceutical to treat an anxiety, depression or psychotic disorder.

Another aspect of the invention disclosed herein relates to methods for treating a patient suffering from an anxiety, depression or psychotic disorder, by co-administering a once-a-day formulation of lithium carbonate (such as an amount from 150 mg to about 2100 mg), and an atypical antipsychotic. In preferred embodiments, the method is carried out using a single oral dosage formulation comprising a sustained-release lithium carbonate in amount from 300 mg to 1200 mg, and the atypical antipsychotic, the formulation being administered as a single dosage unit or multiple dosage units having corresponding fractions of the total daily dose of each active agent. In alternative embodiments of this aspect of the invention, the active agents are used in amounts which would otherwise be subtherapeutic if each agent was used as single active agent therapy for the condition being treated. Exemplary, non-limiting, atypical antipsychotics include clozapine, olanzapine, risperidone, sertindole, quetiapine, and ziprasidone. WO9962522 mentions using lithium in combination with atypical antipsychotics, but gives no details of these combinations. Dosages are indicated as merely having the lithium component set at 600 to 2100 mg per day. U.S. Pat. No. 5,837,701 mentions formulations having lithium carbonate in combination with one of imipramine, trifluoroperazine, or haloperidol, but only in further combination with all of gamma butyric acid, phenylalanine, an antioxidant, folic acid, and nicotinamide.

### BRIEF DESCRIPTION OF THE DRAWING

Not Applicable

### DETAILED DESCRIPTION OF THE INVENTION

### I. Overview

The present invention relates to combinatorial therapies for treating anxiety, depression or psychotic conditions. These diseases or disorders include, but are not limited to, single episodic or recurrent major depressive disorders, dysthymic disorders, depressive neurosis, neurotic depression, melancholic depression, atypical depression, anxiety and phobias, seasonal affective disorder, bipolar disorders, manic depression, unipolar depression, schizophrenia, schizophreniform diseases, acute mania, schizoaffective disorders, and depression with psychotic features. In addition, the present invention is suitable for the treatment of attention-deficit hyperactivity disorder. Furthermore, the present invention is also useful in the treatment of other conditions for which the second drug is indicated.

### II. Definitions

The following defined terms are generally to be used to construe the present invention. However, where the specific statements made indicate a different meaning in a particular context, that meaning shall prevail in that context.

The term “administering” means prescribing or providing medication in a dosage form and amount.

As used herein, the term “depression” includes depressive disorders, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, seasonal affective disorder, or bipolar disorders or manic depression, for example, bipolar I disorder, bipolar II disorder and cyclothymic disorder.

Other mood disorders encompassed within the term “depression” include dysthymic disorder with early or late onset and with or without atypical features; dementia of the Alzheimer's type, with early or late onset, with depressed mood; vascular dementia with depressed mood, disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

By “unipolar depression” or “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, stroke). Generally, unipolar depression is more severe than dysthymia.

The term “anxiety disorders” includes, but is not limited to obsessive-compulsive disorder, psychoactive substance anxiety disorder, post-traumatic stress disorder, generalized anxiety disorder, anxiety disorder NOS, and organic anxiety disorder. Anxiety disorders include panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders. “Generalized anxiety” is typically defined as an extended period (e.g. at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep. “Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterized by an anxiety about being in places or situations from which escape might be difficult or embarrassing or in which help may not be available in the event of a panic attack. Agoraphobia may occur without history of a panic attack A “specific phobia” is characterized by clinically significant anxiety provoked by feared object or situation. Specific phobias include the following subtypes: animal type, cued by animals or insects; natural environment type, cued by objects in the natural environment, for example storms, heights or water, blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterized by clinically significant anxiety provoked by exposure to certain types of social or performance circumstances. Social phobia may also be referred to as social anxiety disorder.

Other anxiety disorders encompassed within the term “anxiety” include anxiety disorders induced by alcohol, amphetamines, caffeine, cannabis, cocaine, hallucinogens, inhalants, phencyclidine, sedatives, hypnotics, anxiolytics and other substances, and adjustment disorders with anxiety or with mixed anxiety and depression.

Anxiety may be present with or without other disorders such as depression in mixed anxiety and depressive disorders. The compositions of the present invention are therefore useful in the treatment of anxiety with or without accompanying depression.

The term “psychotic disorder” includes, for example, schizophrenia, schizophreniform diseases, acute mania, schizoaffective disorders, and depression with psychotic features. The titles given these conditions represent multiple disease states. The following list illustrates a number of these disease states, many of which are classified in the Diagnostic and Statistical Manual of Mental Disorders, 4th Edition, published by the American Psychiatric Association (DSM). The DSM code numbers for these disease states are supplied below, when available, for the convenience of the reader: Paranoid Type Schizophrenia 295.30; Disorganized Type Schizophrenia 295.10; Catatonic Type Schizophrenia 295.20; Undifferentiated Type Schizophrenia 295.90; Residual Type Schizophrenia 295.60; Schizophreniform Disorder 295.40; Schizoaffective Disorder 295.70; Schizoaffective Disorder of the Depressive Type; and Major Depressive Disorder with Psychotic Features 296.24, 296.34.

By “attention-deficit hyperactivity disorder” or “ADHD” is meant a behavioral disorder characterized by a persistent and frequent pattern of developmentally inappropriate inattention, impulsivity, and hyperactivity. Indications of ADHD include lack of motor coordination, perceptual-motor dysfunctions, EEG abnormalities, emotional lability, opposition, anxiety, aggressiveness, low frustration tolerance, poor social skills and peer relationships, sleep disturbances, dysphoria, and mood swings (“Attention Deficit Disorder,” The Merck Manual of Diagnosis and Therapy (17th Ed.), eds. M. H. Beers and R Berkow, Eds., 1999, Whitehouse Station, N.J.).

By “treating” is meant the medical management of a patient with the intent that a cure, amelioration, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

The term “agonist” refers to a compound that mimics the action of natural transmitter or, when the natural transmitter is not known, causes changes at the receptor complex in the absence of other receptor ligands.

The term “antagonist” refers to a compound that binds to a receptor site, but does not cause any physiological changes unless another receptor ligand is present.

The term “ligand” refers to a compound that binds at the receptor site.

The term “pharmaceutically acceptable salt” when used in connection with a drug shall mean acid addition salts of basic drugs and salts of bases with acidic drugs. The particular salt of the drug may be any one which is pharmaceutically acceptable. For acid addition salts, these include, without limitation, acetic, benzene-sulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic and the like. For acidic drugs, the salts are typically, without limitation, alkali metal salts (such as sodium, potassium, etc.), alkaline earth metal salts (such as calcium and magnesium), and ammonium salts. The ordinary skilled pharmaceutical chemist will be able to select appropriate salts from these and many others well known in the art and still be within the scope of the present invention.

### III. Exemplary Formulations

A. Lithium

One embodiment of the present invention relates to combinations of stand-alone lithium formulations and stand alone formulations of other active agents. For these embodiments, any known dosage form of the lithium and the respective second agents are suitable. All that is necessary is that therapeutic levels of each active agent be present within the body at the same time or that if the individual active agent is subtherapeutic (if it were monotherapy) then the combination of the invention be synergistic so that the cotherapy is effective in treating the condition in question. Generally, this condition is met when the drugs are administered at the same time. It is also met at some point during the first treatment day when the lithium and the other active agent are administered separately, and such condition is usually met thereafter until one of the drugs (lithium or the other therapeutic agent) is discontinued. Thus, for such purposes, the lithium formulation and the second drug formulation(s) may be administered together, sequentially, or at any other point within the day (i.e., up to 12 hours apart).

Those of ordinary skill will be able to locate the various known dosage forms by consultation of the various pharmacopeias, formularies, and other general and patent literature related thereto. In this embodiment, non-limiting commercially available lithium formulations in the US as of the filing date of the present application include the following: ESKALITH 300 mg (GlaxoSmithKline), ESKALITH CR 450 mg (GlaxoSmithKline), LITHOBID 300 mg (JDS Pharmaceuticals), Lithium Carbonate Capsules USP 150 mg, 300 mg, and 600 mg (Able Laboratories), Lithium Carbonate Capsules 300 mg (Apotex), Lithium Carbonate Capsules 150 mg, 300 mg, and 600 mg (Roxane), Lithium Carbonate Capsules 150 mg and 300 mg (West Ward), Lithium Carbonate Extended Release Tablets 300 mg (Able Laboratories), Lithium Carbonate Extended Release Tablets 450 mg (Barr), Lithium Carbonate Extended Release Tablets 300 mg and 450 mg (Roxane), Lithium Carbonate Extended Release Tablets 450 mg (West Ward), Lithium Carbonate Extended Release Tablets 300 mg (Pfizer), Lithium to Carbonate Tablets 300 mg (Roxane), and Lithium Citrate Syrup equivalent to 300 mg of lithium carbonate/5 ml (Morton Grove) and Lithium Citrate Syrup equivalent to 300 mg of lithium carbonate/5 ml (Roxane). Use of other novel formulations of lithium salts can be used as long as the lithium component of the invention is delivered within the manner set forth in the present invention.

One of the difficulties with lithium therapy is its low therapeutic index on the one hand, and the need to ensure constant therapeutically useful concentrations, below the toxicity levels, on the other. An appropriate dosing regimen can be obtained with the preparations commercially available at present, carrying out two to three daily administrations; however, the multiple times per day repeat dosings from immediate release formulations (and even current twice daily dosing formulations) tends to result in fluctuations in blood levels that peak and trough outside the therapeutic window (above the suitable maximum level and below the minimum therapeutic level, respectively). Therefore, in preferred embodiments, the present invention utilizes a once daily delivery formulation for the lithium component. When separate formulations are used or when fixed combinations are used with other drugs which are also in a once daily delivery format, the lithium total daily dose can be administered once daily and achieve the desired control over the lithium blood levels. However, when the lithium compound is in a fixed combination with another drug where the other drug is not in a once-daily suitable format, using fractions of the total daily dose, multiple times a day, can still achieve the benefits of cotherapy and better control over the lithium blood level fluctuation, although not as much control over such fluctuation as is achieved from a true once daily product. In this context, for example, a once daily lithium formulation in fixed combination with an immediate release formulation of a second drug which would need to be administered three times a day can be administered in accordance with the present invention as follows: ⅓ the total daily dose of each component is present in a single dose of fixed combination (which can be in one of more dosage units of the fixed combination as desired) with the lithium component being in once daily format. At about 8 hours past the first dose, the second dose is administered, and then 8 hours later the third dose. At the fourth dose, one is close to approaching a steady state level of lithium and the other agent. Each subportion of the daily dose of lithium overlaps the others so that after a few regular fractional dosings, the appropriate blood levels are reached and maintained. A more rapid approach to the appropriate steady state levels of lithium in this context may be achieved by adding a small dose of immediate release lithium to the first and second dosings with the fractional subportions of the present invention.

It should be noted that any of the dosage forms described may contain the entire dose to be administered at any one particular time or any subfraction thereof so that if a once daily dosing regimen is desired, one may administer a single once daily formulation having the entire daily dose, or multiple dosage units having subfractions of the once daily dose (i.e. 2 dosage forms each having ½ the total daily dose of lithium in a once daily formulation and optionally containing ½ the total daily dose of the second drug also in a once daily formulation).

In highly preferred embodiments, the methods and compositions of the present invention utilize a controlled release formulation of lithium carbonate suitable for once daily administration so as to better ensure a constant plasma concentration over approximately 24 hours at levels compatible with the established safety margins.

Suitable, non-limiting once daily formulations of lithium carbonate for use in the present invention can be achieved in accordance with the formulations disclosed in US 2002/0172727 and US2004/0013746, both of which are incorporated herein in their entirety by reference as well as obvious variants thereof. As a non-limiting example of these disclosures, once daily formulations of lithium carbonate are obtained using coated granules of lithium carbonate, where the lithium carbonate is granulated with a binder and then coated with a suitable coating material. A flow agent is added at various points in the coating process to prevent the granules from clumping together. These granules may be used (a) alone as is, or, if desired, (b) further coated with immediate release lithium carbonate or (c) combined with additional uncoated lithium carbonate granules, or (d) combined with lithium carbonate powder. In US2004/0013746, polyvinylpyrrolidone is highlighted as the binder and ethylcellulose as the coating material, with talc used as the flow agent. The granules can be coated with known techniques, preferably with the fluidized bed technique by spraying a solution of the coating agent (ethylcellulose) in ethanol, acetone and water on the active ingredient granules. The coated granules are then incorporated in a suitable carrier for oral administration. The coated granules, incorporated in a suitable carrier, preferably in hard-gelatin capsules, ensure a constant release rate of the active ingredient over approximately 24 hours, without giving rise to the plasma absorption peaks usually observed after multiple times per day lithium formulations. US 2002/0172727 discloses preparation of once daily lithium products in a similar fashion while indicating a broader range of the excipients that can be used. While these references discuss the use of lithium carbonate, the teachings thereof can be applied to any pharmaceutically acceptable salt of lithium for use in the present invention. Because of the large daily dose of lithium salt (up to 2000 mg per day or more), and the limits in the volume of medicament a patient is reasonably willing to take, most once daily formulations of a lithium salt will need to be primarily the lithium salt. Thus, the generally (patient) acceptable once daily formulations of lithium salts for the present invention requires a formulation having at least 50% lithium salt, preferably at least about 60% lithium salt, more preferably at least about 70% lithium salt, more preferably at least about 80% lithium salt, still more preferably at least 89% lithium salt, yet more preferably at least 90% lithium salt, even more preferably at least 91% lithium salt, and can be as much as up to 99.9% but likely up to about 99% and usually up to about 98%, even more preferably up to about 97%, and most probably up to about 95% lithium salt. However, nothing limits the scope of the present invention from lower or higher percentages of lithium salt. As the lithium salt % decreases in the formulation, it may become necessary that the total daily dose be administered by more than one dosage unit at a time. Such once daily dosing with multiple dosage units having fractions of the total daily dose are perfectly suitable for use in the present invention. Other sustained and controlled release formulations of lithium salts may be utilized in the present invention but are limited to being administered to once daily, twice daily, three times daily, or four times daily in accordance with the particular delivery properties of the particular formulation in question. Exemplary sustained or controlled release lithium salt formulations include, without limitation, those disclosed in US 2004/0241252; U.S. Pat. No. 6,365,196; U.S. Pat. No. 4,264,513; U.S. Pat. No. 6,667,060; U.S. Pat. No. 6,143,353; U.S. Pat. No. 5,639,476; U.S. Pat. No. 5,580,578; U.S. Pat. No. 5,286,494; U.S. Ser. NO. 445,829; U.S. Pat. No. 5,133,974; U.S. Pat. No. 5,122,384; EP 93538; and CA 1012887, all of which are incorporated herein by reference.

The pharmacokinetics of the particularly favorable embodiments allow for administration of a single daily dose, increasing patient compliance. The envisaged dosages for the lithium carbonate active ingredient can range from a minimum of 150 mg (and lower for pediatric doses) to a maximum of 2000 mg per day, depending on a number of factors such as the severity of the disorder to treat, the age, weight and conditions of the patient. In certain preferred embodiments, the lithium component is provided in suitable dosages for prophylaxis or acute treatment, i.e. 150 mg to 1200 mg/day in the prophylaxis and up to 2000 mg/day in the acute treatment of states of mania. In certain preferred embodiments, the lithium is provided in an amount ranging from 300 mg to 900 mg, and even more preferably 600 mg to 900 mg. Dosages of 300, 450 or, preferably, 600 mg of lithium carbonate in a single daily dose ensure optimal results. In certain embodiments, because of the synergistic nature of the combinations of the inventions, lower doses of the lithium component than would be required in monotherapy with lithium may be utilized and even subtherapeutic doses ranging from as little as 1/10 the minimum monotherapy therapeutic dose, preferably from ⅛ the minimum monotherapy therapeutic dose, more preferably from ⅕ the minimum monotherapy therapeutic dose, still more preferably from ¼ the minimum monotherapy therapeutic dose, even more preferably from ⅓ the minimum monotherapy therapeutic dose, still more preferably ½ the minimum monotherapy therapeutic dose, yet more preferably from ⅔ the minimum monotherapy therapeutic dose, and most preferably ¾ the minimum monotherapy therapeutic dose may be successfully used. Thus where the minimum therapeutic total daily dose of lithium in monotherapy is for example 500 mg, the present invention contemplates minimum dosages that are as low as about 50 mg per day for that indication when used in the co-therapy of the present invention. Those of ordinary skill will be able to determine the minimum therapeutic dosages of lithium in various indication s in the literature. Notwithstanding any specific determination of minimum amounts of lithium for use in the present invention, the present invention contemplates total daily doses of lithium as small as 25 mg/day, 30 mg/day, 40 mg/day, 50 mg/day, 75 mg/day, 100 mg/day, 125 mg/day, 150 mg/day, 200 mg/day, 250 mg/day, 300 mg/day, 350 mg/day, 400 mg/day, 450 mg/day, 500 mg/day, 550 mg/day and 600 mg/day.

B. Antidepressants

(i) 5-HT2 Receptor Antagonists

Nefazodone (SERZONE®), 2-[3-[4-(3-chlorophenyl)-1-piperazinyl]-propyl-]5-ethyl-2,4-dihydro-4-(2-phenoxy-ethyl)-3H-1,2,4-triazol-3-one, is an antidepressant chemically unrelated to tricyclic or tetracyclic antidepressants and the selective serotonin reuptake inhibitors in current use. Nefazodone has at least two activities in vivo. It blocks serotonin 5-HT₂ receptors at low doses and reversibly inhibits serotonin re-uptake at higher doses. It does not inhibit monoamine oxidase and exhibits decreased anticholinergic, antihistamine, alpha-adrenergic and sedative activity relative to tricyclic antidepressants. At low doses (e.g., 20-40 mg/day in adult humans), nefazodone selectively inhibits 5-HT₂. However, clinically useful effects typically require much higher doses (e.g., 300-600 mg/day) at which serotonin reuptake is also inhibited. Davis et al., Drugs 1997, 53, 608-636; Sanchez et al., Cell Mol. Neurobiol. 1999, 19, 467-489.

Nefazodonoids useful in the present methods and compositions include compounds that inhibit 5-HT₂ receptor activity and have a structure of the following formula:

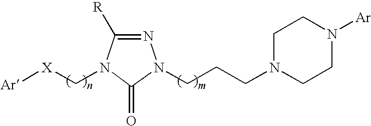

wherein, as valence and stability permit,

Ar and Ar′ represent, independently, substituted or unsubstituted aryl groups;

X represents O or S, preferably O;

R represents a hydroxyl or a substituted or unsubstituted lower alkyl group, lower alkoxy, lower acyloxy, aralkoxy, or aracyloxy group;

n represents an integer from 24, preferably 3; and

m represents an integer from 0-2, preferably 1.

In certain embodiments, Ar is phenyl group, and is unsubstituted or, preferably, is substituted with 1-5 substituents selected from halogen and CF₃ groups.

In certain embodiments, R represents an ethyl group optionally substituted with a hydroxyl group, oxo group, or a lower acyloxy group. In embodiments wherein R represents hydroxyl, the formula is considered to include the triazoledione tautomer.

Examples of compounds which fall within, or can be modified with a hydroxyl, oxo, or other substituent to R in order to fall within, the above formula can be found in U.S. Pat. Nos. 4,338,317, 4,386,091, 4,613,600, 5,116,852, 4,575,555, and 4,487,773, and PCT publication WO 00/661128.

In certain embodiments, a nefazodonoid has a structure of the following formula:

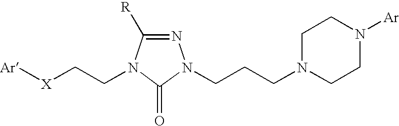

wherein, as valence and stability permit,

Ar and Ar′ represent, independently, phenyl rings, either unsubstituted or substituted with from 1-3 groups selected from halogen and CF₃ groups;

X represents O or S, preferably O; and

R represents a hydroxyl or a C₁-C₃ alkyl group, either unsubstituted or substituted with a hydroxyl, oxo, or lower acyloxy group.

In certain embodiments, Ar is unsubstituted or, preferably, is substituted with a halogen or CF₃ group.

In certain embodiments, Ar′ is unsubstituted or substituted with a halogen or CF₃, group.

In certain embodiments, R represents an ethyl group optionally substituted with a hydroxyl group. In embodiments wherein R represents hydroxyl, the formula is considered to include the triazoledione tautomer.

Nefazodone undergoes fairly rapid metabolism in the body, resulting in the formation of several metabolic derivatives. Of these, hydroxynefazodone and its oxonefazodone and triazoledione metabolic derivatives retain some or all of nefazodone's activity against 5-HT₂ receptors. Accordingly, any of these compounds as well as any other metabolic derivatives of nefazodone that retain some or all of nefazodone's 5-HT₂ inhibitory activity, and pharmaceutically acceptable salts of any of these, may be employed in the compositions and methods of the invention, and are considered to be nefazodonoids as the term is used herein.

(ii) Serotonin Reuptake Inhibitors (SRI).

The measurement of a compound's activity as an SSRI is now a standard pharmacological assay. Wong, et al., Neuropsychopharmacology 8, 337-344 (1993). Many compounds, including those discussed at length above, have such activity, and no doubt many more will be identified in the future. In the practice of the present invention, it is intended to include reuptake inhibitors which show 50% effective concentrations of about 1000 nM or less, in the protocol described by Wong supra. Serotonin reuptake inhibitors include, but are not limited to:

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine and showed that their activity as serotonin reuptake inhibitors is similar to each other. In this document, the word “fluoxetine” will be used to mean any acid addition salt or the free base, and to include either the racemic mixture or either of the R and S enantiomers;

Duloxetine, N-methyl-3-(1-naphthalenyloxy)-3-(2-thienyl)propanamine-, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent;

Milnacipran (N,N-diethyl-2-aminomethyl-1-phenylcyclopropanecarboxam-ide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake;

Citalopram, 1-[3-(dimethylamino)propyl]-1-(4-fluorophenyl)-1,3-dihy-dro-5-isobenzofurancarbonitrile, is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-(trifluoromethyl)-phenyl]-1-pentanone O-(2-aminoethyl)oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluo-rophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985);

Sertraline, (1S-cis)-4-(3,4-dichlorophenyl)-1,2,3,4-tetrahydro-N-me-thyl-1-naphthylamine hydrochloride, is a serotonin reuptake inhibitor which is marketed as an antidepressant. It is disclosed by U.S. Pat. No. 4,536,518;

To illustrate, the SRI can be venlafaxine or a derivative thereof. For instance, the SRI can be a compound represented in the following formula, or a pharmaceutically acceptable salts thereof:

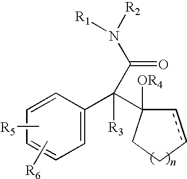

wherein

R₁ is hydrogen or alkyl of 1 to 6 carbon atoms;

R₂ is alkyl of 1 to 6 carbon atoms;

R₃ is hydrogen or alkyl of 1 to 6 carbon atoms;

R₄ is hydrogen, alkyl of 1 to 6 carbon atoms, formyl, or alkanoyl of 2 to 7 carbon atoms;

R₅ and R₆ are independently hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkylamino of 1 to 6 carbon atoms, dialkylamino in which each alkyl group is of 1 to 6 carbon atoms, alkanamido of 2 to 7 carbon atoms, halo, trifluoromethyl, or, when taken together, methylene dioxy; and

n is one of the integers 0, 1, 2, 3 or 4.

The nontricyclic compound venlafaxine, chemically named (±)-1-[2-(dimethylamino)-1-(4-methoxyphenyl)ethyl]-cyclohexanol, is an antidepressant which has been studied extensively and which is described in, for example, U.S. Pat. No. 4,761,501 and Pento, J. T. Drugs of the Future 13(9):839-840 (1988).

Venlafaxine includes active derivatives of venlafaxine. The term “derivative” includes metabolites. Venlafaxine derivatives include: O-desmethylvenlafaxine and the single enantiomers of the two compounds.

In certain preferred embodiments, the venlafaxine compound is provided in optically pure form, such as optically pure (−)—N-desmethylvenlafaxine, chemically named (−)-1-[2-(methylamino)-1-(4-methoxyphenyl)ethyl]cyclohexanol; optically pure (−)-N,N-didesmethylvenlafaxine, chemically named (−)-1-[2-(amino)-1-(4-methoxyphenyl)ethyl]cyclohexanol; optically pure (−)—O-desmethylvenlafaxine, chemically named (−)-1-[2-(dimethylamino)-1-(4-phenol)ethyl]cyclohexanol; optically pure (−)—N,O-didesmethylvenlafaxine, chemically named (−)-1-[2-(methylamino)-1-(4-phenol)ethyl]cyclohexanol; and optically pure (−)—O-desmethyl-N,N-didesmethylvenlafaxine, chemically named chemically named (−)-1-[2-(amino)-1-(4 phenol)ethyl]cyclohexanol.

In other embodiments, the SRI compound is an optically pure derivative of (+)-venlafaxine, such as (+)—O-desmethylvenlafaxine. U.S. Pat. No. 6,197,828 provides additional examples of derivatives of (+)-venlafaxine.

In preferred embodiments, the SRI is a selective serotonin reuptake inhibitor (SSRI). SSRIs include fluoxetinoids, sertraline (ZOLOFT), citalopram (CELEXA), paroxetine (PAXIL), and fluvoxamine (LUVOX), cericlamine, femoxetine, ifoxetine, cyanodothiepin, and litoxetine. The terms such as “sertraline,” “citalopram,” “paroxetine,” and “fluvoxamine” include active derivatives and metabolites, such as the desmethyl metabolites of norfluoxetine, desmethylsertraline, and desmethylcitalopram.

Preferred SSRIs are fluoxetinoids and citalopram (and its derivatives). More preferred SSRIs are fluoxetinoids.

Fluoxetinoids useful in the present methods and compositions include compounds that inhibit serotonin reuptake and have structures of the following formula:

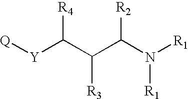

wherein, as valence and stability permit,

R₁, independently for each occurrence, represents H or lower alkyl, preferably H or Me;

R₂, R₃, and R₄ each independently represent H, methyl, substituted or unsubstituted phenyl, or substituted or unsubstituted phenylmethyl, such that exactly one of R₂, R₃, and R₄ is a substituted or unsubstituted phenyl, or substituted or unsubstituted phenylmethyl;

Y represents O, S, or —S(O)₂—, preferably O;

Q represents a substituted or unsubstituted aryl or heteroaryl ring, including polycyclic ring systems.

In certain embodiments, at least one occurrence of R₁ represents hydrogen.

In certain embodiments, R₂ and R₃ are selected from H and Me, preferably H, and R₄ represents a substituted or unsubstituted phenyl ring.

In certain embodiments, Q is a substituted or unsubstituted phenyl ring.

Examples of compounds which fall within the above formula can be found in U.S. Pat. Nos. 4,902,710, 4,824,868, 4,692,469, 4,626,549, 4,584,404 and 4,314,081.

In certain embodiments, a fluoxetinoid has a structure of the following formula:

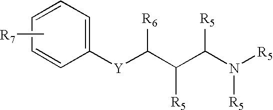

wherein, as valence and stability permit,

R₅, independently for each occurrence, represent H or Me;

R₆ represents a substituted or unsubstituted phenyl ring, preferably unsubstituted;

Y represents O, S, or —S(O)₂—, preferably O; and

R₇ represents from 1-5 substituents selected from halogen, lower alkyl, lower alkenyl, lower alkoxy, substituted or unsubstituted phenyl, and CF₃.

In certain embodiments, at least one occurrence of R₅ bound to N is a hydrogen. In certain embodiments, R₆ represents an unsubstituted phenyl group.

In certain embodiments, R₇ represents from 1-2 substituents selected from halogen and CF₃.

Fluoxetine is metabolized far more slowly, with the primary metabolic derivative being norfluoxetine, which is similar to fluoxetine in selectivity and potency. Any combination of these compounds, racemic or enriched for either enantiomer, and pharmaceutically acceptable salts thereof may be employed in the methods and compositions described herein, and any one of these compounds is included in the term ‘fluoxetinoids’ as the term is used herein.

In certain embodiments, the SSRI is sertraline or a derivative thereof. For instance, the SSRI can be a compound represented in Formula, or a pharmaceutically acceptable salts thereof:

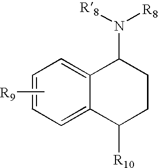

wherein

R₈ is selected from the group consisting of hydrogen and normal alkyl of from 1 to 3 carbon atoms;

R′₈ is normal alkyl of from 1 to 3 carbon atoms;

R₉ is selected from the group consisting of hydrogen, fluoro, chloro, bromo, trifluoromethyl and alkoxy of from 1 to 3 carbon atoms;

R₁₀ is

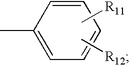

R₁₁ and R₁₂ are each independently selected from the group consisting of hydrogen, fluoro, chloro, bromo, trifluoromethyl, alkoxy of from 1 to 3 carbon atoms and cyano, with at least one of R₁₁ and R₁₂ being other than hydrogen.

U.S. Pat. Nos. 4,536,518, 4,940,731, 4,962,128, and 5,130,338 describe sertraline and various derivatives and formulations thereof which can be used in the subject formulation and methods. Sertraline derivatives include N-desmethylsertraline.

In certain preferred embodiments, the compound is, as appropriate, the cis-isomeric base of the above formula. The term “cis-isomeric” refers to the relative orientation of the N(R′₈) R₈ and R₁₀ moieties on the cyclohexene ring (i.e. they are both oriented on the same side of the ring). Because both the 1- and 4-carbons of the formula are asymmetrically substituted, each cis-compound has two optically active enantiomeric forms denoted (with reference to the 1-carbon) as the cis-(1R) and cis-(1S) enantiomers. The preferred embodiment is the (1S) enantiomer, e.g., cis-(1S)—N-methyl-4-(3,4-dichlorophenyl)-1,2,3,4-tetrahydro-1-naphthalenamine and its pharmaceutically acceptable acid addition salts.

In certain embodiments, the SSRI is paroxetine or a derivative thereof. For instance, the SSRI can be a compound represented in Formula, or a pharmaceutically acceptable salts thereof:

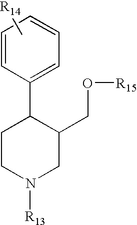

wherein

R₁₃ represents hydrogen or an alkyl group of 1-4 carbon atoms, and

R₁₄ represents hydrogen, alkyl having 1-4 carbon atoms, C1-6 alkoxy, C1-6 trifluoroalkyl (preferably, trifluoromethyl), hydroxy, halogen, methylthio, or C1-6 aryl(C1-6) alkyloxy (e.g., phenyl(C1-6)alkyloxy and benzyl(C1-6)alkyloxy), and

R₁₅ represents an alkyl or alkynyl group having 1-4 carbon atoms, or a phenyl group optionally substituted by C1-4 alkyl, C1-6 alkylthio, C1-6 alkoxy, halogen, nitro, acyl amino, methylsulfonyl or methylenedioxy, or represents tetrahydronaphthyl.

In certain preferred embodiments, the SSRI is a compound represented in Formula, or a pharmaceutically acceptable salts thereof:

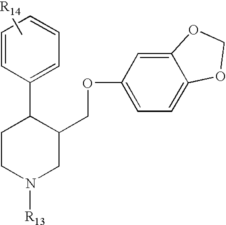

wherein R₁₃ represents hydrogen or an alkyl group of 1-4 carbon atoms, and R₁₄ is a halogen. In certain preferred embodiments, R₁₃ is a fluorine. Of particularly therapeutic effect is the (−) form of a compound of formula I, wherein R¹ is hydrogen and the fluorine is in para position.

The synthesis of paroxetine and of the acid addition salts thereof is described, inter alia, in U.S. Pat. No. 4,007,196 to Christensen et al. and U.S. Pat. No. 4,721,723 to Barnes et al. Derivative of paroxetine are also described in PCT publication WO035910.

In still other embodiments, the SSRI is citalopram or a derivative thereof. For instance, the SSRI can be a compound represented in Formula, or a pharmaceutically acceptable salts thereof:

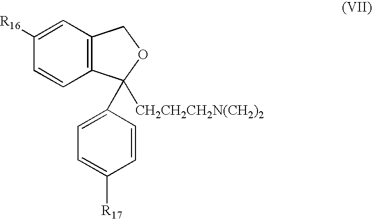

wherein R₁₆ and R₁₇ are each independently represent a halogen, a trifluoromethyl group, a cyano group or —C(═O)—R₁₈, wherein R₁₈ is an alkyl radical with from 1-4 C-atoms inclusive.

Citalopram was first disclosed in DE 2,657,271 corresponding to U.S. Pat. No. 4,136,193. This patent publication describes the preparation of citalopram by one method and outlines a further method which may be used for preparing citalopram Methods of preparing the individual enantiomers of citalopram are disclosed in U.S. Pat. No. 4,943,590, such as (+)-1-(3-Dimethylaminopropyl)-1-(4′-fluorophenyl)-1,3-dihydroisobenzofuran-5-carbonitrile. Citalopram derivatives include desmethylcitalopram and didesmethylcitalopram, and the single enantiomers of all three compounds.

In yet another embodiment, the SSRI is fluvoxamine or a derivative thereof. For instance, the SSRI can be a compound represented in Formula, or a pharmaceutically acceptable salts thereof:

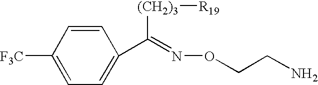

wherein R₁₉ represents a cyano group, a cyanomethyl group, a methoxymethyl group or an ethoxymethyl group. Fluvoxamine and other oxime ethers are disclosed in U.S. Pat. No. 4,085,225.

The magnitude of prophylactic or therapeutic doses of an SRI and a nefazodonoid will, of course, vary with the nature and the severity of the condition to be treated and the route of administration, as well as the age, weight and response of the individual patient. In general, the daily dose range of fluoxetine or norfluoxetine administered as part of the conjoint therapy contemplated herein lies within the range of from about 1 mg to about 100 mg per day, preferably about 5 mg to about 60 mg per day, and most preferably from about 10 mg to about 40 mg per day, in single or divided doses. In general, the daily dose range of nefazodone or hydroxynefazodone administered in conjoint therapy as contemplated herein lies within the range of from about 1 mg to about 100 mg per day, preferably about 5 mg to about 60 mg per day, and most preferably from about 10 mg to about 40 mg per day, in single or divided doses. On the other hand, it may be necessary to use dosages outside these limits in some cases.

Any suitable route of administration may be employed for providing the patient with effective dosages of an SRI or a nefazodonoid. For example, oral, rectal, parenteral, transdermal, subcutaneous, intramuscular, inhalation and the like may be employed. Dosage forms include tablets, troches, dispersions, suspensions, solutions, capsules, patches and the like.

The pharmaceutical compositions of the present invention comprise an SRI or a nefazodonoid as an active ingredient or a pharmaceutically acceptable salt thereof, and to may also contain a pharmaceutically acceptable carrier and optionally other therapeutic ingredients.

Since fluoxetines and nefazodones are generally basic, salts may be prepared using pharmaceutically acceptable non-toxic acids, including inorganic and organic acids. Such acids include acetic, benzene-sulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic and the like. Particularly preferred are hydrobromic, hydrochloric, phosphoric and sulfuric acids.

The compositions include compositions suitable for oral, rectal, parenteral (including subcutaneous, intramuscular, and intravenous), although the most suitable route in any given case will depend on the nature and severity of the condition being treated. The most preferred route of the present invention is oral. They may be conveniently presented in unit dosage form and prepared by any of the methods well known in the art of pharmacy.

In the case where an oral composition is employed, a suitable dosage range of fluoxetine is, e.g., from about 1 mg to about 50 mg of fluoxetine per day, preferably from about 5 mg to about 45 mg per day and most preferably from about 10 mg to about 40 mg per day, and a suitable dosage range of nefazodone is, e.g., from about 1 mg to about 120 mg of fluoxetine per day, preferably from about 10 mg to about 100 mg per day and most preferably from about 20 mg to about 80 mg per day.

(iii) NK-3 Antagonist

Exemplary NK-3 antagonists are described in US Patent Publication 20040006135.

(iv) NK-1 Receptor Antagonist

Exemplary NK-1 antagonists are described in US Patent Publication 20040006135.

(v) PDE IV Inhibitor

Exemplary PDE IV inhibitors are described in US Patent Publication 20040001895.

(vi) Neuropeptide Y5 Receptor Antagonists

Exemplary Neuropeptide Y5 receptor antagonists are described in PCT Publication WO03051397.

(vii) D4 Receptor Antagonist

Exemplary PDE IV inhibitors are described in US Patent Publication 20020094986.

(viii) 5HT1D Receptor Antagonist

Exemplary PDE IV inhibitors are described in US Patent Publication 20020049211.

(ix) Miscellaneous

Suitable norepinephrine reuptake inhibitors of use in conjunction with in the present invention include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof. Other norepinephrine reuptake inhibitors of use in conjunction with the present invention include, without limitation, atomoxetine, nisoxetine, and reboxetine.

Suitable monoamine oxidase inhibitors of use in conjunction with the present invention include: isocarboxazid, phenelzine, tranylcypromine and selegiline, and pharmaceutically acceptable salts thereof. Suitable reversible inhibitors of monoamine oxidase of use in conjunction with the present invention include: moclobemide, and pharmaceutically acceptable salts thereof.

Suitable CRF antagonists of use in conjunction with the present invention include those compounds described in International Patent Specification Nos. WO 94/13643, WO 94/13644, WO 94/13661, WO 94/13676 and WO 94/13677.

Other antidepressants of use in conjunction with the present invention include adinazolam, alaproclate, amineptine, amitriptyline/chlordiazepoxide combination, atipamezole, azamianserin, bazinaprine, befuraline, bifemelane, binodaline, bipenamol, brofaromine bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metaprarine, metralindole, mianserin, milnacipran, minaprine, mirtazapine, montirelin, nebracetam, nefopam, nialamide, nomifensine, norfluoxetine, orotirelin, oxaflozane, pinazepam, pirlindone, pizotyline, ritanserin, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometapine, and pharmaceutically acceptable salts thereof, and St. John's wort herb, or Hypericum perforatum, or extracts thereof.

Suitable classes of anti-anxiety agent of use in conjunction with the present invention also include benzodiazepines. Suitable benzodiazepines of use in conjunction with the present invention include: alprazolam, chlordiazepoxide, clonazepam, chlorazepate, diazepam, halazepam, lorazepam, oxazepam and prazepam, and pharmaceutically acceptable salts thereof.

In addition to benzodiazepines, other suitable classes of anti-anxiety agent are nonbenzodiazepine sedative-hypnotic drugs such as zolpidem; mood-stabilizing drugs such as clobazam, gabapentin, lamotrigine, loreclezole, oxcarbamazepine, stiripentol and vigabatrin; and barbiturates.

Suitable 5-HT_{1A} receptor agonists or antagonists of use in conjunction with the present invention include, in particular, the 5-HT_{1A} receptor partial agonists buspirone, flesinoxan, gepirone and ipsapirone, and pharmaceutically acceptable salts thereof. An example of a compound with 5-HT_{1A} receptor antagonist/partial agonist activity is pindolol.

Another class of anti-anxiety agent of use in conjunction with the present invention are compounds having muscarinic cholinergic activity. Suitable compounds in this class include muscarinic cholinergic receptor agonists such as those compounds described in European Patent Specification Nos. 0709093, 0709094 and 0773021, and PCT Publication WO 96/12711.

Another class of anti-anxiety agent of use in conjunction with the present invention are compounds acting on ion channels. Suitable compounds in this class include carbamazepine, lamotrigine and valproate, and pharmaceutically acceptable salts thereof.

C Anticonvulsants/Antiepileptics

Antiepileptic and anticonvulsants contemplated as the second component include, but are not limited to, phenyloins (phenyloin, mephenyloin and ethotoin), barbiturates (phenobarbital, mephobarbital, and primidone), iminostilbenes (carbamazepine and oxcarbamazepine), succinimides (ethosuximide), valproic acid, oxazolidinediones (trimethadione) and other antiseizure agents (gabapentin, lamotrigine, acetazolamide, felbamate, and γ-vinyl GABA).
Carbamezepine, 5H-dibenz [b,f]azepine-5-carboxamide is an anticonvulsant and analgesic marketed for trigeminal neuralgia; U.S. Pat. No. 2,948,718 (herein incorporated by reference in their entirety), discloses carbamezepine.
Valproic Acid, 2-propylpentanoic acid or dispropylacetic acid is a well known antiepileptic agent which dissociates to the valproate ion in the gastrointestinal tract; various pharmaceutically acceptable salts are disclosed in U.S. Pat. No. 4,699,927.
Lamotrigine, 6-(2,3-dichlorophenyl)-1,2,4-trizine-3,5-diamine is an antiepileptic drug indicated as adjunctive therapy in the treatment of partial seizures in adults with epilepsy. Lamotrigine is disclosed in U.S. Pat. No. 4,486,354.
Gabapentin, 1-(aminomethyl)cyclohexane acetic acid, is an anticonvulsant indicated as adjunctive therapy in the treatment of partial seizures with and without secondary generalization in adults with epilepsy. Gabapentin is described in U.S. Pat. Nos. 4,024,175 and 4,087,544.
Topiramate, 2,3:4,5-di-O-(1-isopropylidine)-3-D-fructopyranose sulphamate is an antiepileptic and disclosed in U.S. Pat. No. 4,513,006.

D. Combination of Once-a-Day Lithium with Atypical Antipsychotics

Another aspect of the invention relates to the combination of a once-a-day formulation of lithium carbonate, e.g., a pharmaceutical composition containing an amount of lithium carbonate from 150 mg to 900 mg, preferably from 300 mg to 900 mg (such as coated granules described above), along with an atypical antipsychotic, for the treatment of depression, anxiety, or a psychotic condition.

The essential feature of an atypical antipsychotic is less acute extrapyramidal symptoms, especially dystonias, associated with therapy as compared to a typical antipsychotic such as haloperidol. While conventional antipsychotics are characterized principally by D2 dopamine receptor blockade, atypical antipsychotics show antagonist effects on multiple receptors including the 5HT₂ₐ and 5HT_{2c} receptors and varying degrees of receptor affinities. See Meltzer in Neuropsychopharmacology: The Fifth Generation of Progress 2002, pp 819-831; and Baldessarini and Tarazi in Goodman & Gilman's The Pharmacological Basis of Therapeutics 10th Edition, 2001, pp 485. Atypical antipsychotic drugs are also commonly referred to as serotonin/dopamine antagonists, reflecting the influential hypothesis that greater affinity for the 5HT₂ receptor than for the D2 receptor underlies “atypical” antipsychotic drug action or “second generation antipsychotic” drugs.

Clozapine, the prototypical atypical antipsychotic, differs from the typical antipsychotics with the following characteristics: (1) greater efficacy in the treatment of overall psychopathology in patients with schizophrenia nonresponsive to typical antipsychotics; (2) greater efficacy in the treatment of negative symptoms of schizophrenia; and (3) less frequent and quantitatively smaller increases in serum prolactin concentrations associated with therapy (Beasley, et al., Neuropsychopharmacology, 14(2), 111-123, (1996)). Atypical antipsychotics include, but are not limited to:
Olanzapine, 2-methyl-4-(4-methyl-1-piperazinyl) 10H-thieno[2,3-b][1-, 5] benzodiazepine, is a known compound and is described in U.S. Pat. No. 5,229,382;
Clozapine, 8-chloro-11-(4-methyl-1-piperazinyl)-5H-dibenzo[b,e][1,4-] diazepine, is described in U.S. Pat. No. 3,539,573;
Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663;
Sertindole, 1-[2-[4-[5-chloro-1-(4-fluorophenyl-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945;
Quetiapine, 5-[2-(4-dibenzo[b,f][1,4]thiazepin-11-yl-1-piperazinyl)-ethoxy]ethanol, and its activity in assays which demonstrate utility in the treatment of schizophrenia are described in U.S. Pat. No. 4,879,288. Certain preferred embodiments, Quetiapine is provided as its (E)-2-butenedioate (2:1) salt; and

Ziprasidone, 5-[2-[4-(1,2-benzoisothiazol-3-yl)-1-piperazinyl]ethyl-]-6-chloro-1,3-dihydro-2H-indol-2-one, and especially its hydrochloride monohydrate. The compound is described in U.S. Pat. Nos. 4,831,031 and 5,312,925.

The preparation of such compounds is fully described in the aforementioned patents and publications.

Suitable pharmaceutically acceptable salts of the compounds of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium, ammonium, magnesium, and calcium salts thereof.

E. Formulations

The single active agent standalone formulations of each of the above second active agents (and the manner of making the same) is adequately set forth in the relevant patents and literature discussed above, each of which is incorporated herein by reference (in their entirety).

The lithium salt and second drug components may be co-administered in combination (including by coformulation) by oral, parenteral (e.g., intramuscular, intraperitoneal, intravenous or subcutaneous injection, or implant), nasal, vaginal, rectal, sublingual, transdermal, or topical routes of administration and can be formulated in dosage forms appropriate for each route of administration.

Preferably the compositions according to the present invention are in unit dosage forms such as tablets, pills, capsules, powders, granules, solutions or suspensions, or suppositories, for oral, parenteral or rectal administration, by inhalation or insufflation or administration by trans-dermal patches or by buccal cavity absorption wafers.

In one embodiment of the invention when the cotherapy is accomplished by administering different formulations of the lithium salt and the second active agent by solid dosage forms such as tablets or capsules, the formulations will generally include the principle active agent and at least one pharmaceutically acceptable carrier; and, in addition, may have one or more of typical excipients such as binders, lubricants, disintegrants, colorants, polishing agents, coating agents, etc. The principal active ingredients are independently mixed with pharmaceutical carriers, e.g. conventional tableting ingredients such as corn starch, lactose, sucrose, sorbitol, talc, stearic acid, magnesium stearate, dicalcium phosphate or gums, and other pharmaceutical diluents to form a first pre-blend in which the active agent is homogenously distributed. This pre-blend, or the raw active agent, may be compressed directly into a core, granulated with a granulating solution or dissolved in a solvent for spray drying onto an different (generally inert) core. Immediate release formulations may then be blended with other excipients such as lubricants, glidents, disintegrants, etc. to form a second blend. The second blend can be filled into capsules and used as is, or compressed into tablets with or without additional coatings. For extended release or sustained release formulations, generally the preblend may include a polymeric material to give sustained release properties to the formulations or the granules may be coated with a solution of a sustained release coating solution, or the compressed tablets (which may be microtablets) may be coated with a sustained release coating. Alternatively, sustained release delivery may be achieved by formulations known in the art as oral-osmotic formulations (a.k.a. OROS), a technology originally developed by Alza Corporation, in which a tablet formulation having either a highly water soluble high osmotic strength producing component therein (which may or may not be the active agent) or a water swellable polymer is coated with a semi water-permeable membrane with a hole drilled therein. Once exposed to an aqueous environment, the water permeates the membrane and dissolves the highly soluble high osmotic pressure producing agent resulting in additional water being imbibed or swells the water-swellable polymer. The water influx builds pressure and forces the dissolved solution out of the tablet through the pre-drilled hole. Particulars of oral osmotic formulation construction are well known in the art and those of ordinary skill will be able to construct appropriate modifications thereof. When referring to these pre-blend compositions as homogeneous, it is meant that the active ingredient is dispersed evenly throughout the composition so that the composition may be readily subdivided into equally effective unit dosage forms such as tablets, pills and capsules. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Other non-limiting excipients which may be used in the present invention include: talc, croscarmelose sodium, silicon dioxide, magnesium stearate, dibasic calcium phosphate, hydroxypropylmethylcellulose, hydroxypropylcellulose, polyethyleneglycols, polysorbates, sodium starch glycolate, crospovidone, polyvinylpyrrolidone, hydroxymethylcellulose, titanium dioxide, iron oxides, starch, glycerin, lactose, sucrose, fructose, mannose, sodium stearyl fumarate, sorbitol, mannitol, gelatin, silicones, carnauba wax, pharmaceutical glaze, etc. In addition, those excipients which are contained in the commercially available formulated products of the active agents discussed herein as of the filing date of this application as disclosed in published labeling or the 2005 Edition of the PDR are deemed included herein as suitable formulation excipients. A much more extensive list will be known to those of ordinary skill.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Compositions of the present invention may also be presented for administration in the form of transdermal patches using conventional technology. The compositions may also be administered via the buccal cavity using, for example, a rapidly dissolving tablet and/or absorption wafers.

When fixed combination dosage forms are desired, they may be prepared in a number of manners with techniques generally known in the art. For example, the individual active agents may be blended together to form an active blend and the active blend is then processed as above for the single active agent formulation. Alternatively, the two active agents may be blended with different portions of the same or different components that will go into the “pre-blend” mentioned above and these two partial pre-blends may be blended together to form a dual agent pre-blend which is then processed further in accordance with the single agent pre-blend mentioned above. Alternatively, each active agent may be blended and granulated independently and the granules blended in the appropriate ratios for further processing in accordance with the above disclosure concerning the single active agent formulations. In another embodiment, one active agent and binder may form a continuous phase in which the other active or other active agent pre-blend or granule is dispersed. Typically in this embodiment, the lithium salt would form the continuous phase due to the high daily dosage needed relative to the daily dosage of the other active agent. Still other variations include bi-layer tablets in which the two active agents are physically contained in their own separate formulations which are compressed together in layers with or without binder adhering the two layers together. Capsule formulations may contain granules of blends of the active agents, blends of granules of each of the active agents, or microtablets of compressed granules of these variations. A multitude of optional variants on the theme will be apparent to those of ordinary skill in the art.

Especially preferred variations are fixed combinations suitable for once daily administration, whether or not actually administered in a once daily regimen. The ratio of the active agents is set so as to result in (a) therapeutic levels of each active agent if used alone or (b) subtherapeutic levels of one or both active agents when used alone, but due to synergies of the combination, the combination as a whole reaches therapeutic effectiveness. Thus, under variant (a) the ratio of actives varies from the single active agent minimum effective dose of active agent 1: the single active agent maximum tolerated dose of active agent 2 up to and including the single active agent minimum effective dose of active agent 2: the single active agent maximum tolerated dose of active agent 1 on an active agent basis. Such a ratio insures both active agents to be within the therapeutic window of each of the actives when the total daily dose is administered. Under variant (b), the ratios of the drugs may vary more widely since one or both of the drugs may be administered in a dose which is subtherapeutic if it were used at that dose in monotherapy for the same condition. Thus, overall, the ratio of the active agents depends on both the individual active agent and the synergies resulting from the combination therapy. In addition, some of the active agents indicated in the present invention may need to be titrated on a patient by patient basis. Thus, for obtaining specific fixed combination ratios and daily dosage amounts for variant (b) above, one may utilize any of the ratios in variant (a) above and titrate down from the total daily dose in (a) until efficacy is lost and then increase the daily dose slightly. Alternatively, for ratios that are outside of those found in variant (a) above, one may administer separate dosage forms of single active agent formulations and titrate one or both for optimal results and then set this as the fixed combination ratio and dosage. The above ratio and daily dosage amount identification procedure may also be used in variants where separate single entity dosage forms are utilized in the invention as well as when multiple administrations per day variations are utilized (whether or not fixed combinations).

To help insure appropriate patient compliance with dosing of coadministration of single active agent dosage forms, it is preferable to package the appropriate number of dosage units of each type (and if fixed combinations to package the appropriate number of dosage units) in a blister pack so that the patient will either self administer or have administered the correct dosage of each active agent. Thus, one non-limiting example is a blister package having two capsules containing 600 mg each of once daily lithium carbonate and one tablet of 10 mg fluoxetine hydrochloride as a single unit of use package for a patient requiring 1200 mg/day lithium and 10 mg/day fluoxetine. Multiple unit of uses may be packaged together as separate blister packs or on the same blister pack with adequate labeling thereon to distinguish one day's dose from another. Other adequate package variations will be well known to those of ordinary skill. For low dose (ie subtherapeutic when that active agent would be used in monotherapy) variations of the present invention, one example would be a similar blister package having two capsules each containing 600 mg lithium carbonate and 1 capsule containing 5 mg of fluoxetine hydrochloride as a single unit of use. A third variation of the present invention would be a blister pack having one capsule containing 150 mg of lithium carbonate and one capsule containing 5 mg of fluoxetine hydrochloride. Other combinations of dosage forms of a lithium salt and another active agent in accordance with the present invention will be apparent to those of ordinary skill in the art.

It will be appreciated to those skilled in the art that reference herein to treatment extends to prophylaxis (prevention) as well as the treatment of the noted diseases/disorders and symptoms. Because the specific diagnosis of depression and/or anxiety in a particular patient may be difficult, the patient may benefit from the prophylactic administration of a subject compound in accordance with the present invention.

It should also be noted that patients presenting with psychiatric symptoms, especially depression, may actually be unrecognized manic-depressives. In such patients, the administration of antidepressant medication alone may precipitate a manic attack. Thus, the addition of lithium to antidepressant therapy is highly desirable. However, in such cases where the manic phase of a manic depressive is not overtly manifest, lower amounts of lithium are more desirable than when such a condition is overtly noticeable. The present invention adequately suppresses precipitating a manic attack in a sub-clinical manic depressive patient being treated with antidepressants and thus offers a safety benefit to such patients. Furthermore, many antidepressant, especially SSRIs, have become associated with elevated risk of suicidal tendencies. Lithium coadminsitration with such agents (even low dose lithium) helps to prevent such tendencies.

### Examples

The following non-limiting Examples are presented only to exemplify various embodiments of the invention and do not limit it in any fashion.

### Example 1

Once daily lithium carbonate beads are prepared in accordance with Example 1 of US 2004/0013746 and filled into #2 capsules so as to contain 300 mg lithium carbonate. The following commercially available SSRI's are obtained in finished dosage forms from the manufacturers thereof as listed in the Physician's Desk Reference 2005 Edition.

TABLE I

| | |
|---|---|
| LEXAPRO (escitalopram oxalate) | 10 mg and 20 mg |
| ZOLOFT (sertraline hydrochloride) | 25 mg, 50 mg, and 100 mg |
| PROZAC (fluoxetine hydrochloride) | 10 mg, 20 mg, and 40 mg |
| CELEXA (citalopram hydrobromide) | 10 mg, 20 mg, and 40 mg |
| PAXIL (paroxetine hydrochloride) | 10 mg, 20 mg, 30 mg, and 40 mg |
| LUVOX (fluvoxamine maleate) | 25 mg, 50 mg, and 100 mg |

Each of the 6 products in the table is indicated for once daily administration in the current labeling of the commercially available dosage forms listed. Unit of use blister packs are prepared containing two capsules of the 300 mg lithium carbonate once daily capsules and one dosage unit selected from Table I to prepare 18 different units of use blister packs containing 600 mg of a once daily lithium carbonate and 10-100 mg of an SSRI in Table I.

### Example II

Is 101721 Two of the 300 mg lithium carbonate capsules prepared for use in Example I and one dosage unit selected from the SSRIs in Table I are selected for preparing fixed combinations of lithium carbonate and the SSRI. Tablets are lightly broken to powder, capsule are emptied. The tablet or capsule powder from the SSRI selected and the granules of lithium carbonate from the two lithium carbonate capsules are blended together. The blend is (a) repackaged in 2 appropriate sized capsules or (b) compressed into 1 or 2 tablets. The repackaged capsules or tablets so formed are packaged in blister packs or bottles.

### Example III

Two of the 300 mg lithium carbonate capsules prepared for use in Example I are emptied and combined with the amount of the pure active agent indicated in Table II and repackaged in two capsules each containing 300 mg lithium carbonate and ½ the amount of the SSRI indicated in Table I.

### Example IV

A dry blend of the amount of pure SSRI active agent indicated in Table I above is blended with 50-75 mg of lactose, 25-50 mg of pregelatinized starch and 1-5 mg of magnesium or calcium stearate. The blend is then blended with the granular contents of two 300 mg lithium carbonate capsules and repackaged in two appropriately sized capsules each containing 300 mg lithium carbonate and 12 the amount of the SSRI indicated in Table I.

### Example V

The amount of pure SSRI indicated in Table I above is blended with 40-60 mg of dicalcium phosphate, 15-30 mg of Avicel, and 1-8 mg of magnesium or calcium stearate. This blend is then slugged/compacted to from a dry granulation. The dry granulation is blended with the contents of two of the 300 mg lithium carbonate capsules prepared for use in Example I and repackaged in two appropriately sized capsules each containing 300 mg lithium carbonate and ½ the amount of the SSRI indicated in Table I.

### Example VI

The amount of pure SSRI indicated in Table I above is blended with 30-70 mg lactose and 70-100 mg pregelatinized starch. The blend is granulated with an aqueous solution of polyvinylpyrrolidone such that the granules will contain from 5-10 mg of polyvinylpyrrolidone. The granules are dried and 1-10 mg of magnesium or calcium stearate is added thereto along with the contents of two 300 mg lithium carbonate capsules prepared fro use in Example I. This blend is then repackaged in two appropriately sized capsules each containing 300 mg lithium carbonate and ½ the amount of the SSRI indicated in Table I.

Alternatively, the SSRI wet granules are dried and compressed into minitablets such that ½ of the minitablets can be and are added to the contents of each of two 300 mg lithium carbonate capsules that had been prepared for use in Example I. If necessary, due to volume requirements, the capsule contents of the lithium carbonate granules and the is minitablets are repackaged in larger capsules so that each contains 300 mg lithium carbonate and ½ the amount of the SSRI indicated in Table I.

### Example VII

A coating solution of the amount of SSRI indicated in Table I in aqueous alcohol containing 10-30 mg of hydroxypropylmethylcellulose and 5-10 mg sodium laurylsulfate is prepared. The contents of two 300 mg lithium carbonate capsules prepared for use in Example I are emptied and spray coated with the coating solution and dried. The thus coated lithium carbonate granules are repackaged into in two appropriately sized capsules each containing 300 mg lithium carbonate and ½ the amount of the SSRI indicated in Table I.

Alternatively, instead of coating the lithium carbonate granules, the coating solution is used to coat sugar spheres. The so coated sugar spheres are then blended with the contents of two 300 mg lithium carbonate capsules prepared for use in Example I and this blend is repackaged into in two appropriately sized capsules each containing 300 mg lithium carbonate and ½ the amount of the SSRI indicated in Table I.

### Example VIII

Examples I-VII are repeated except that an amount of the granules produced for use in Example I equivalent to 50 mg of the lithium carbonate are used with the equivalent of one daily dosage amount of the second active agent.

### Example IX

Examples I-VII are repeated except 5 mg of the second active agent set forth in Table I above are used in for a single daily dose of cotherapy according to the present invention.

### Example X

Examples I-VII are repeated except that that 50 mg of the lithium carbonate granules produced for use in Example I and 5 mg of the second active agent set forth in Table I above are used for a single daily dose of cotherapy according to the present invention.

### Example XI

Examples I-VII are repeated except that that the second active agent in Example I is replaced with 1 mg, 5 mg, 10, 20, 40, or 50 mg of an active agent set forth below: acetazolamide, adinazolam, alaproclate, alprazolam, amineptine, amitriptyline, amoxapine, atomoxetine, atipamezole, azamianserin, bazinaprine, befuraline, bifemelane, binodaline, bipenamol, brofaromine bupropion, buspirone, carbamazepine, caroxazone, cericlamine, chlorazepate, chlordiazepoxide, cianopramine, cimoxatone, citalopram, clemeprol, clobazam, clomipramine, clonazepam, clovoxamine, clozapine, dazepinil, deanol, demexiptiline, desipramine, diazepam, dibenzepin, dothiepin, doxepin, droxidopa, enefexine, estazolam, ethotoin, ethosuximide, etoperidone, felbamate, femoxetine, fengabine, fezolamine, flesinoxan, fluotracen, gabapentin, gepirone, halazepam, hydroxynefazodone, idazoxan, imipramine, indalpine, indeloxazine, iprindole, ipsapirone, isocarboxazid, lamotrigine, levoprotiline, litoxetine, lofepramine, lorazepam, loreclezole, maprotiline, medifoxamine, mephobarbital, mephenyloin, metaprarine, metralindole, mianserin, milnacipran, minaprine, mirtazapine, moclobemide montirelin, ebracetam, nefazodone, nefopam, nialamide, nisoxetine, nomifensine, norfluoxetine, nortriptyline, olanzapine, orotirelin, oxaflozane, oxazepam, oxcarbamazepine, oxonefazodone, phenelzine, phenobarbital, phenyloin, pinazepam, pirlindone, pizotyline, prazepam, primidone, protriptyline, quetiapine, risperidone; reboxetine, ritanserin, selegiline, sercloremine, sertindole, setiptiline, sibutramine, stiripentol, sulbutiamine, sulpiride, teniloxazine, thozalinone, thymoliberin, tianeptine, tiflucarbine, tofenacin, tofisopam, toloxatone, tomoxetine, tranylcypromine, trimethadione, trimipramine, valproate, veralipride, vigabatrin, zimelidine, viqualine, ziprasidone, zometapine, and γ-vinyl GABA.

1. A method for treating a patient suffering from an anxiety, depression or psychotic disorder, comprising co-administering an effective amount of a first component which includes a lithium salt, in combination with an effective amount of a second component selected from the group consisting of a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, a sedative-hypnotic drug, an atypical antipsychotic, and mixtures or combinations thereof.

2.-26. (canceled)

27. A kit comprising
a. a mood-stabilizing lithium formulation, and a second drug selected from the group consisting of a serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, a sedative-hypnotic drug, and an atypical antipsychotic,
b. instructions for co-administering the lithium formulation and the second drug in the method of claim 1.

28. (canceled)

29. A single oral dosage formulation of a sustained lithium carbonate and a second component selected from the group consisting of a serotonin reuptake inhibitor, a 5HT₂ receptor antagonist, an anticonvulsant, a norepinephrine reuptake inhibitor, an a-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, a sedative-hypnotic drug, and an atypical antipsychotic.

30.-34. (canceled)

35. A method of preventing or reducing the incidence of suicidal tendencies associated with the use of a psychoactive drug comprising administering a lithium salt as cotherapy with said psychoactive drug wherein said psychoactive drug is selected from the group consisting of serotonin reuptake inhibitor, a 5HT₂ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, a sedative-hypnotic drug, and an atypical antipsychotic.
